(19) United States
(12) Patent Application Publication
(10) Pub. No.: US 2025/0214968 A1
(43) Pub. Date: Jul. 3, 2025

(54) Novel Salt of GLP-1 Receptor Agonist Compound, Preparation Method Thereof and Pharmaceutical Composition Comprising Thereof

(71) Applicant: Ildong Pharmaceutical Co., Ltd., Seoul (KR)

(72) Inventors: Hong Chul YOON, Hwaseong-si, Gyeonggi-do (KR); Kyung Mi AN, Hwaseong-si, Gyeonggi-do (KR); Joon Tae PARK, Hwaseong-si, Gyeonggi-do (KR); Jung Woo LEE, Hwaseong-si, Gyeonggi-do (KR); Jung-Eun PARK, Hwaseong-si, Gyeonggi-do (KR); Jae Eui SHIN, Hwaseong-si, Gyeonggi-do (KR); Min Whan CHANG, Hwaseong-si, Gyeonggi-do (KR); Mi Young LEE, Hwaseong-si, Gyeonggi-do (KR); Han Na SEO, Hwaseong-si, Gyeonggi-do (KR); Won Je SEONG, Hwaseong-si, Gyeonggi-do (KR); Hyeong Jun LEE, Hwaseong-si, Gyeonggi-do (KR); In-Gyu JE, Hwaseong-si, Gyeonggi-do (KR)

(73) Assignee: Ildong Pharmaceutical Co., Ltd., Seoul (KR)

(21) Appl. No.: 18/850,107

(22) PCT Filed: Mar. 24, 2023
(86) PCT No.: PCT/KR2023/003988

§ 371 Date: Sep. 24, 2024

(30) Foreign Application Priority Data
Mar. 25, 2022 (KR) 10-2022-0037344

(51) Int. Cl. C07D 401/14; A61K 31/496

(52) U.S. Cl. CPC C07D 401/14; A61K 31/496

(57) ABSTRACT

The present disclosure relates to a GLP-1 receptor agonist compound, and a novel salt thereof having excellent in vivo solubility, stability, and bioavailability, a preparation method thereof, and a pharmaceutical composition comprising the same.

### TECHNICAL FIELD

The present disclosure relates to a novel salt of a glucagon-like peptide-1 receptor (GLP-1R) agonist compound exhibiting improved physical properties and excellent bioavailability in terms of stability, solubility, and the like, a preparation method thereof, and a pharmaceutical composition comprising the same.

### BACKGROUND ART

It is well known based on large-scale clinical studies that strict blood sugar control toward a normal blood sugar level in diabetes treatment is important for preventing various complications caused by the diabetes. A candidate compound that may lower the blood sugar by strongly stimulating the secretion of insulin includes a hormone referred to as glucagon-like peptide-1 (GLP-1). GLP-1 was first discovered in 1985 as an incretin hormone secreted by L-cells in ileum and colon. GLP-1 increases insulin secretion by acting on a receptor referred to as GLP-1R (glucagons-like peptide-1 receptor). GLP-1 is secreted via stimulation by absorbed nutrients or blood sugar levels. Diabetes treatment using GLP-1 has advantages that hypoglycemia does not occur because insulin is secreted depending on the glucose concentration. In addition, this hormone is known to be effective in reducing movement of an upper digestive system and suppressing appetite, and to proliferate existing beta cells of the pancreas.

Currently, various GLP-1 analogs having resistance to the DPP-4 enzyme that destroys GLP-1 in the blood have been developed and are being used as treatments for type 2 diabetes. Since these GLP-1 analogues have a considerably longer half-life compared to GLP-1, they have the advantage of maintaining the hypoglycemic effect for a long time, but there is a problem of low medication convenience in that these GLP-1 analogues are not able to be administered orally and should be used in the form of injections. Therefore, in recent years, some studies have been conducted to discover a small-molecule GLP-1R agonist capable of being administered orally and develop the agonist as a therapeutic agent for diabetes.

In this regard, improvements in physicochemical properties such as solubility, stability, non-hygroscopicity, and the like, together with improvement in bioavailability, and reduction in toxicity are required for small-molecule GLP-1R agonists.

Under this background, the present inventors found that a tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid as a GLP-1 receptor agonist compound had excellent physicochemical properties, high purity, and excellent bioavailability compared to other commonly used pharmaceutically acceptable salt compounds, and completed the present disclosure.

### DISCLOSURE OF INVENTION

### Technical Problem

An object of the present disclosure is to provide a novel salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid.

In particular, the present disclosure provides a novel salt having excellent physicochemical and/or pharmaceutical properties such as hygroscopicity, thermal stability, solubility, and the like, to have excellent formulation processability and storage stability, and excellent bioavailability, a preparation method thereof, and a pharmaceutical composition comprising the same.

### Solution to Problem

In one general aspect, the present disclosure provides a novel salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid, a preparation method thereof, and a pharmaceutical composition comprising the same, as an active ingredient.

Hereinafter, each detailed description is provided below.

### Novel salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid

For the above purpose, the present disclosure provides a tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid.

The tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid may be represented by the following Chemical Formula I:

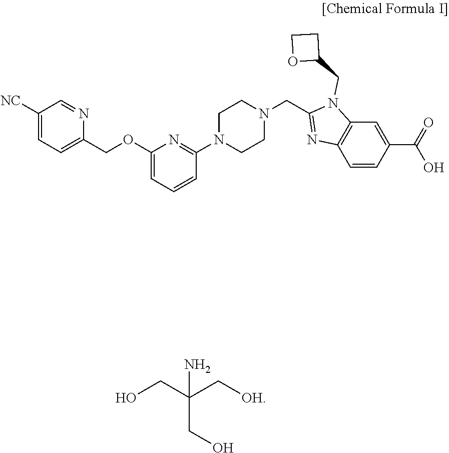

The novel salt according to the present disclosure exhibits excellent physicochemical and pharmacokinetic properties in various aspects such as thermal stability, in vivo solubility, bioavailability, and the like.

The tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid of the present disclosure may have a thermogravimetric analysis (TGA) pattern showing a weight loss of less than 1.0 wt %, 0.9 wt %, 0.8 wt %, 0.7 wt %, 0.6 wt %, 0.5 wt %, 0.4 wt %, 0.3 wt %, or 0.2 wt % at 170° C. or less. Specifically, the thermogravimetric analysis (TGA) pattern of FIG. 2 may be shown.

The tromethamine salt (specifically, crystalline form thereof) of the present disclosure is characterized by having an endothermic transition peak value at 174 to 204° C. when a heating rate is 10° C./min in a differential scanning calorimetry (DSC) graph, and preferably, is characterized by having an endothermic transition peak value at 179 to 199° C., more preferably, at 184 to 194° C., and more preferably at 189±2° C.

Further, the tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid of the present disclosure may show the differential scanning calorimetry of FIG. 2.

In addition, the novel tromethamine salt of the present disclosure is preferably in a crystalline form.

The crystalline form of the tromethamine salt of the present disclosure may comprise, in an X-ray powder diffraction (XRPD) graph, at least three diffraction peaks at 2-theta (20) angle values selected from the group consisting of 4.03±0.2, 8.09±0.2, 10.04±0.2, 15.07±0.2, 15.71±0.2, 17.90±0.2, 19.60±0.2, 22.07±0.2, 24.92±0.2, and 25.39±0.2.

More specifically, the crystalline form of the tromethamine salt of the present disclosure may comprise, in an X-ray powder diffraction (XRPD) graph, diffraction peaks at 2-theta (2θ) angles of 4.03±0.2, 8.09±0.2, 10.04±0.2, 15.07±0.2, 15.71±0.2, 17.90±0.2, 19.60±0.2, 22.07±0.2, 24.92±0.2, and 25.39±0.2.

Still more specifically, the crystalline form of the tromethamine salt of the present disclosure may further comprise, in the XRPD graph, any one or more diffraction peaks at 2-theta (2θ) angle values selected from the group consisting of 12.17±0.2, 14.39±0.2, 17.02±0.2, 20.18±0.2, and 21.81±0.2.

In the present disclosure, the tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid, having the aforementioned features, can be named crystalline form 1.

Further, the crystalline form of the tromethamine salt of the present disclosure may further comprise, in the XRPD graph, any one or more diffraction peaks at 2-theta (2θ) angle values selected from the group consisting of 19.80±0.2 and 24.53±0.2.

In the present disclosure, the crystalline form of the tromethamine salt of the present disclosure may further comprise, in the XRPD graph, diffraction peaks at 2-theta (2θ) angle values 19.80±0.2 and 24.53±0.2, the crystalline form may be crystalline form 1A.

Further, the tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid of the present disclosure may exhibit the X-ray powder diffraction spectroscopy pattern of FIG. 1.

In the present disclosure, a novel salt that had never been used in the related art was prepared. Specifically, the tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid may have excellent thermal stability, and stability according to temperature and humidity, thereby being stably maintained without a change in amount over a long period of time. In particular, the tromethamine salt has excellent thermal stability against high temperature, and the like, to exhibit excellent storage stability. Therefore, the raw material of the novel salt of the present disclosure is able to be obtained in high yield and high purity, and the increase in related substances is remarkably low even when stored for a long time, and thus high purity may be maintained for a long period of time.

In addition, the novel salt of the present disclosure may exhibit excellent solubility values under various pH conditions, particularly bio-relevant media conditions, to thereby have high bioavailability, thus achieving excellent pharmacological effects, and may be usefully employed as a new active ingredient of a pharmaceutical composition capable of treating various indications.

According to an embodiment of the present disclosure, when preparing stimulated gastric fluid (SGF), fasted state simulated intestinal fluid (FaSSIF), and fed state simulated intestinal fluid (FeSSIF) and performing a test for measuring solubility and dissolution under conditions close to the in vivo environment, good solubility was shown. In particular, the solubility in FaSSIF was very good in the tromethamine salt. It was confirmed from this finding that the novel salt according to the present disclosure had significantly excellent solubility in vivo and exhibited high bioavailability. In addition, since the novel salt according to the present disclosure has high in vivo solubility to exhibit high levels of exposure in the body, and maintains a high blood concentration, thereby having a remarkable effect of excellent bioavailability.

In addition, since the novel salt of the present disclosure exhibits high bioavailability when administered orally, it is possible to exhibit excellent therapeutic effects even when taken in a small amount, thereby significantly improving the patient's medication compliance.

Further, the novel salt of the present disclosure may have a rapid onset of action and a thermodynamically stable form, and may be very advantageous in processing and storage of pharmaceutical products to achieve easy formulation, and further, may maintain the same state even after the formulation is prepared so that the uniformity in view of the formulation amount may be stably maintained for a long period of time, and thus the novel salt may be easily applied to mass production.

In the present disclosure, the said tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid, crystalline form 1, can be changed to crystalline form 1A upon exposure to atmospheric moisture at a specific relative humidity condition, specifically at 20° C. and 55% RH or higher.

But, crystalline form 1A may return to the crystal form 1 upon desorption by changing the relative humidity conditions at 20° C., less than 55% RH, preferably 20° C., 50% RH or less, more preferably 20° C., 45% RH or less.

The tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid, crystalline form 1, according to the present disclosure is a non-stoichiometric hydrate, and when exposed to moisture in a wide range of atmosphere up to 90% RH, only expansion and contraction of the lattice due to water molecules occur, and there is no collapse of the lattice structure or crystal phase transition during adsorption and desorption. Therefore, even if the crystalline form of the tromethamine according to the present disclosure adsorbs moisture when exposed to a wide range of humidity, it can be reverted to the original crystalline form, which is the crystalline form 1, when desorbed.

### Preparation Method of Tromethamine Salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid

In another general aspect, the present disclosure provides a preparation method of a tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid represented by the following Chemical Formula I:

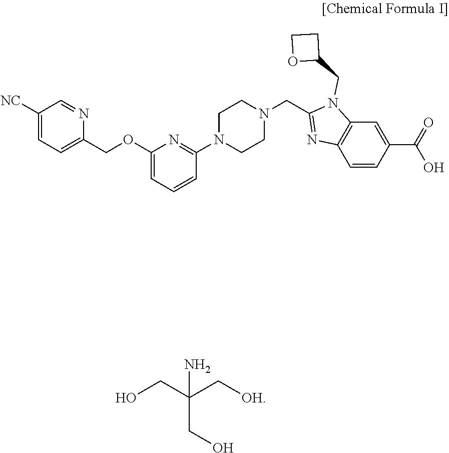

Specifically, the preparation method of the present disclosure may be divided into the following steps:
(1) mixing a compound represented by the following Chemical Formula II with a single organic solvent or a mixed organic solvent and reacting with tromethamine represented by the following Chemical Formula III;

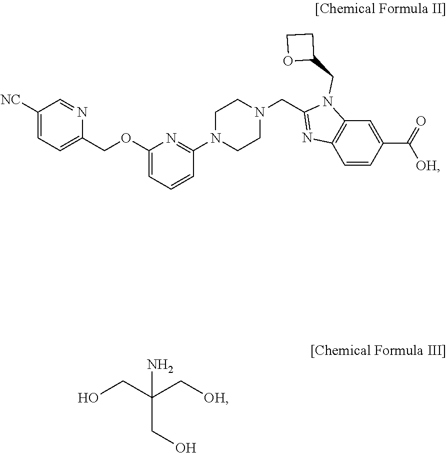

(2) heating and warm-stirring a reaction solution obtained in step (1);
(3) cooling and stirring a reaction solution obtained in step (2); and
(4) filtering and drying a product of step (3).

In the preparation method of the present disclosure, the compound represented by the following Chemical Formula II is(S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid, which may be a free base prepared according to Comparative Example 1 of the present disclosure.

In the preparation method of the present disclosure, the organic solvent used in the reaction in step (1) may be a single organic solvent or a mixture of organic solvents. The organic solvent may be one or a combination of two or more selected from the group consisting of methanol, ethanol, isopropyl alcohol, acetonitrile, n-propanol, acetone, methyl ethyl ketone, methyl acetate, ethyl acetate, tetrahydrofuran, and 2-methyl tetrahydrofuran. Preferably, the organic solvent may be one or a combination of two or more organic solvents selected from the group consisting of methanol, ethanol, isopropyl alcohol, acetonitrile, acetone, and ethyl acetate.

A mixing ratio of the mixed solvent may be 1:1 to 1:20 by volume.

In the present disclosure, tromethamine represented by Chemical Formula III is called tris(hydroxymethyl)aminomethane, and is also referred to as Tris. Thus, as used herein, Tris means tromethamine.

Tromethamine in step (1) is preferably used in an amount of 0.6 to 1.5 equivalents, more preferably in an amount of 0.7 to 1.3 equivalents, and still more preferably in an amount of 0.9 to 1.1 equivalents, based on 1.0 equivalent of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid.

In the preparation method of the present disclosure, the reacting in step (1) may be performed at a temperature of 20 to 80° C., preferably 40 to 70° C., and more preferably 55 to 60° C.

Step (2) may be a step of precipitating a solid precipitate, and step (3) may be a step of separating the solid precipitate produced in step (2), wherein the solid precipitate may be separated by processes commonly performed in the relevant field, such as a principle separation method, and the like.

In the preparation method of a novel salt of the present disclosure, the reaction temperature and time may be adjusted according to the type and amount of the sample and solvent used in the reaction, but are not limited to the above range.

### Pharmaceutical Composition

The present disclosure provides a pharmaceutical composition for preventing or treating metabolic diseases comprising a tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid represented by the following Chemical Formula I;

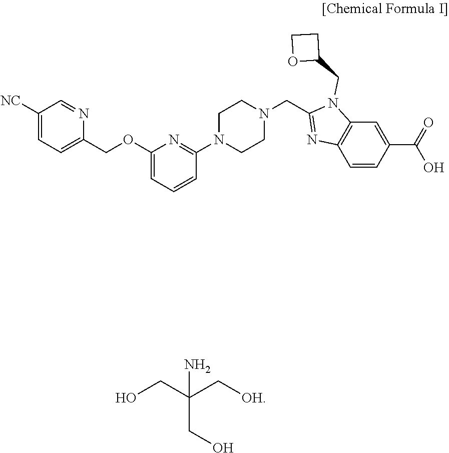

The novel tromethamine salt according to the present disclosure may have excellent stability, thermal stability, and pH stability, and may exhibit excellent in vivo solubility under bio-relevant media conditions close to the in vivo environment, thereby showing excellent pharmacological effects.

The term metabolic disease used herein includes, for example, diabetes (TID and/or T2DM, such as prediabetes), idiopathic TID (type 1b), latent autoimmune diabetes in adults (LADA), early onset T2DM (EOD), youth-onset atypical diabetes (YOAD), maturity onset diabetes of the young (MODY), malnutrition-related diabetes, gestational diabetes, hyperglycemia, insulin resistance, liver insulin resistance, impaired glucose tolerance, diabetic neuropathy, diabetic nephropathy, kidney disease (e.g., acute kidney failure, tubular dysfunction, pro-inflammatory changes to proximal tubule), diabetic retinopathy, adipocyte dysfunction, visceral fat accumulation, sleep apnea, obesity (e.g., hypothalamic obesity and monogenic obesity) and associated co-morbidities (e.g. osteoarthritis and urinary incontinence), eating disorders (e.g., binge eating syndrome, anorexia nervosa, and syndrome of obesity, such as Prader-Willi syndrome and Bardet-Biedl syndrome), weight gain due to use of other drugs (e.g. from use of steroids and antipsychotics), excessive sugar intake, dyslipidemia (including hyperlipidemia, hypertriglyceridemia, increased total cholesterol, high LDL cholesterol, and low HDL cholesterol), hyperinsulinemia, NAFLD (including related diseases such as steatosis, NASH, fibrosis, cirrhosis, and hepatocellular carcinoma), cardiovascular disease, atherosclerosis (including coronary artery disease), peripheral vascular disease, hypertension, endothelial dysfunction, impaired vascular compliance, congestive heart failure, myocardial infarction (e.g., necrosis and apoptosis), stroke, hemorrhagic stroke, ischemic stroke, traumatic brain injury, pulmonary hypertension, restenosis after angioplasty, intermittent claudication, postprandial lipidemia, metabolic acidosis, ketosis, arthritis, osteoporosis, Parkinson's disease, left ventricular hypertrophy, peripheral arterial disease, loss of vision, cataracts, glomerulosclerosis, chronic renal failure, metabolic syndrome, syndrome X, premenstrual syndrome, angina, thrombosis, atherosclerosis, transient ischemic attack, vascular restenosis, impaired glucose metabolism, symptoms of impaired fasting blood sugar, hyperuricemia, gout, erectile dysfunction, skin and connective tissue disorders, psoriasis, foot ulcers, ulcerative colitis, hyper-apo B lipoproteinemia, Alzheimer's disease, schizophrenia, cognitive impairment, inflammatory bowel disease, short bowel syndrome, Crohn's disease, colitis, irritable bowel syndrome, polycystic ovary syndrome, and addiction (e.g., alcohol and/or drug abuse).

Specifically, the metabolic disease may be at least any one selected from the group consisting of diabetes mellitus, idiopathic type 1 diabetes, latent autoimmune diabetes in adults (LADA), early onset type 2 diabetes (EOD), youth-onset atypical diabetes (YOAD), maturity-onset diabetes of the young (MODY), malnutrition-related diabetes, gestational diabetes, hyperglycemia, insulin resistance, liver insulin resistance, impaired glucose tolerance, diabetic neuropathy, diabetic nephrosis, kidney disease, diabetic retinopathy, visceral fat accumulation, sleep apnea, obesity, eating disorders, dyslipidemia, hyperinsulinemia, non-alcoholic fatty liver disease (NAFLD), cardiovascular disease, atherosclerosis, peripheral vascular disease, hypertension, congestive heart failure, myocardial infarction, stroke, hemorrhagic stroke, ischemic stroke, traumatic brain injury, pulmonary hypertension, restenosis after angioplasty, intermittent claudication, postprandial lipidemia, metabolic acidosis, ketosis, arthritis, osteoporosis, Parkinson's disease, left ventricular hypertrophy, peripheral arterial disease, loss of vision, cataracts, glomerulosclerosis, chronic renal failure, metabolic syndrome, Syndrome X, premenstrual syndrome, angina, thrombosis, transient ischemic attack, vascular restenosis, impaired glucose metabolism, symptoms of impaired fasting blood sugar, hyperuricemia, gout, erectile dysfunction, psoriasis, foot ulcers, ulcerative colitis, hyper-apo B lipoproteinemia, Alzheimer's disease, schizophrenia, cognitive impairment, inflammatory bowel disease, short bowel syndrome, Crohn's disease, colitis, irritable bowel syndrome, and polycystic ovary syndrome.

In detail, the non-alcoholic fatty liver disease (NAFLD) may be, for example, at least any one selected from the group consisting of steatosis, non-alcoholic steatohepatitis (NASH), fibrosis, cirrhosis, and hepatocellular carcinoma. Exemplary non-alcoholic fatty liver diseases described above include a category of metabolic diseases having a significant association with various medical diseases excluding the category of alcoholic liver diseases.

In an aspect, the present disclosure provides a pharmaceutical composition comprising: a tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid represented by the following Chemical Formula I, and a pharmaceutically acceptable carrier:

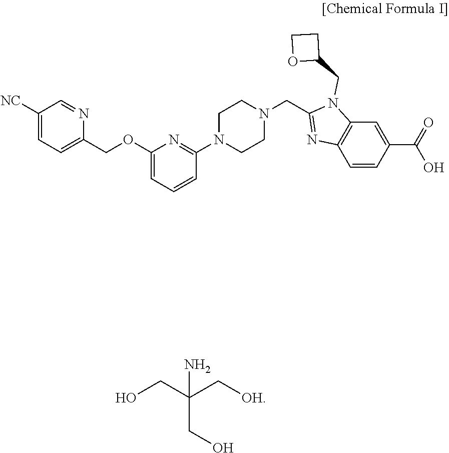

As used herein, the term “pharmaceutically acceptable carrier” includes any and all of physiologically compatible solvents, dispersion media, coatings, antibacterial and antifungal agents, isotonic and absorption delaying agents, and the like.

The composition according to the present disclosure may be in various forms. The composition according to the present disclosure may be in a form of, for example, liquid, semi-solid and solid dosage, such as liquid solutions (e.g., injectable and injectable solutions), dispersions or suspensions, tablets, pills, powders, liposomes and suppositories. The form depends on the intended route of administration and therapeutic purpose thereof.

A typical composition is in the form of injectable and infusible solutions. One mode of administration is a parenteral mode (e.g., intravenous, subcutaneous, intraperitoneal, intramuscular mode).

Oral administration of a solid formulation may be achieved, for example, based on hard or soft capsules, pills, cachets, lozenges or tablets, each containing a prede-termined amount of one or more compounds according to the present disclosure. In some embodiments, oral administration may be achieved based on powder or granular form.

In still other embodiments, oral administration may be achieved in a liquid dosage form. The liquid dosage forms for oral administration include, for example, pharmaceutically acceptable emulsions, solutions, suspensions, syrups, and elixirs containing inert diluents (e.g., water) commonly used in the art.

Other carrier substances and modes of administration known in the pharmaceutical art may be used. The pharmaceutical composition according to the present disclosure may be prepared by any well-known pharmaceutical technique, such as effective formulation and administration procedures.

These formulations may be prepared by conventional methods used for formulation in the art or a method disclosed in the document [see, Remington's Pharmaceutical Science (latest edition), Mack Publishing Company, Easton PA], and may be formulated into various formulations depending on each disease or component.

The composition of the present disclosure may be administered orally or parenterally (for example, intravenous, subcutaneous, intraperitoneal or topical application) according to the desired method, and the dose varies depending on the patient's weight, age, sex, health condition, diet, administration time, administration method, excretion rate, and severity of the disease. The daily dose of the novel salt of the present disclosure is about 0.01 to 500 mg/kg, preferably 0.1 to 100 mg/kg, and may be divided and administered once or several times a day.

The pharmaceutical composition of the present disclosure may further contain at least one active ingredient exhibiting the same or similar medicinal effect in addition to the novel salt.

### Therapeutic Use and Method for Treatment of Metabolic Diseases, and Use Thereof in the Manufacture of a Medicament for Treatment

The present disclosure provides a pharmaceutical composition for the prevention or treatment of metabolic diseases, comprising: a tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid represented by the following Chemical Formula I:

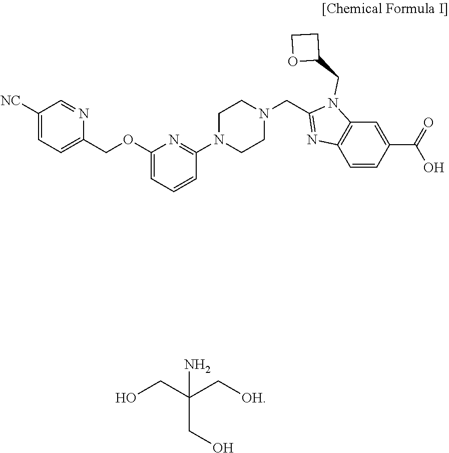

The present disclosure provides a method for treating metabolic diseases, comprising: administering to a subject in need thereof a therapeutically effective amount of the tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid as described above.

The phrase “a subject in need thereof” means mammals including a human, which includes mammals such as humans, monkeys, cattle, horses, dogs, cats, rabbits, rats, and mice.

As used herein, the term “therapeutically effective amount” refers to an amount of the novel salt effective for the prevention or treatment of metabolic diseases, or a pharmaceutical composition comprising the same, and for example, may include, as an amount of the novel salt to be administered to the subject to be treated, any amount of the pharmaceutical composition comprising the above-described salt, to prevent occurrence or recurrence of metabolic diseases, to alleviate symptoms, to inhibit direct or indirect pathological consequences, to prevent metastasis, to reduce the rate of pro-gression, or to alleviate or temporarily ameliorate the condition or to improve the prognosis. In other words, the therapeutically effective amount may be interpreted to encompass all doses in which symptoms of metabolic diseases are improved or cured by the pharmaceutical composition.

The method for preventing or treating metabolic diseases of the present disclosure includes not only treating the diseases themselves before the onset of signs, but also in-hibiting or avoiding signs thereof by administering the above-described salt or the pharmaceutical composition comprising the same. In the management of diseases, a prophylactic or therapeutic dose of a particular active ingredient will vary depending on the nature and severity of the disease or condition and the route by which the active ingredient is administered. The dose and frequency of dose will vary depending on the age, weight and response of individual patients. A suitable dosage regimen may be readily selected by one of ordinary skill in the art considering these factors naturally. In addition, the method for treating metabolic diseases using the pharmaceutical composition of the present disclosure may further include administering a therapeutically effective amount of an additional active agent useful for treating the diseases together with the above-described salt, wherein the additional active agent may exhibit synergistic or auxiliary effects with the above-described salt which is an active ingredient according to the present disclosure.

The present disclosure also provides use of a tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid for the manufacture of a medicament for treatment of metabolic diseases. The above-described salts for the manufacture of a medicament may be mixed with acceptable adjuvants, diluents, carriers, and the like, and may be prepared as a combined preparation with other active agents to have a synergistic action of the active ingredients.

Matters mentioned in the uses, compositions and treatment methods of the present disclosure are applied equally as long as they do not contradict each other.

### Advantageous Effects of Invention

The novel salt according to the present disclosure may have excellent stability to increase the stability of the formulation, and may have improved solubility (particularly, in vivo solubility) and bioavailability to be usefully employed as an active ingredient of a pharmaceutical composition.

### BRIEF DESCRIPTION OF DRAWINGS

The following drawings attached to the present specification illustrate preferred embodiments of the present disclosure, and serve to further understand the technical idea of the present disclosure together with the above-described content of the invention, and thus the present disclosure should not be construed as being limited only to the matters described in these drawings.

FIG. 1 is an X-ray powder diffraction analysis (XRPD) graph of a tromethamine salt compound represented by Chemical Formula I prepared in Example 3 of the present disclosure (crystalline form 1).

FIG. 2 is a thermogravimetric analysis (TGA) pattern analysis graph and a differential scanning calorimetry (DSC) pattern graph of the tromethamine salt compound represented by Chemical Formula I prepared in Example 3 of the present disclosure (crystalline form 1).

FIG. 3 is a thermogravimetric analysis (TGA) pattern analysis graph of a(S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid free base prepared in Comparative Example 1.

FIG. 4 is a differential scanning calorimetry (DSC) pattern analysis graph of the(S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid free base prepared in Comparative Example 1.

FIG. 5 is an X-ray powder diffraction analysis (XRPD) graph of an L-Arginine salt compound of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid prepared in Comparative Example 2.

FIG. 6 is a thermogravimetric analysis (TGA) pattern analysis graph of the L-Arginine salt compound of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid prepared in Comparative Example 2.

FIG. 7 is a differential scanning calorimetry (DSC) pattern analysis graph of the L-Arginine salt compound of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid prepared in Comparative Example 2.

FIG. 8 is an X-ray powder diffraction analysis (XRPD) graph of a meglumine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid prepared in Comparative Example 3.

FIG. 9 is a thermogravimetric analysis (TGA) pattern analysis graph of the meglumine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid prepared in Comparative Example 3.

FIG. 10 is a differential scanning calorimetry (DSC) pattern analysis graph of the meglumine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid prepared in Comparative Example 3.

FIG. 11 is an XRPD graph of a potassium salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid prepared in Comparative Example 4.

FIG. 12 is a TGA pattern analysis graph of the potassium salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid prepared in Comparative Example 4.

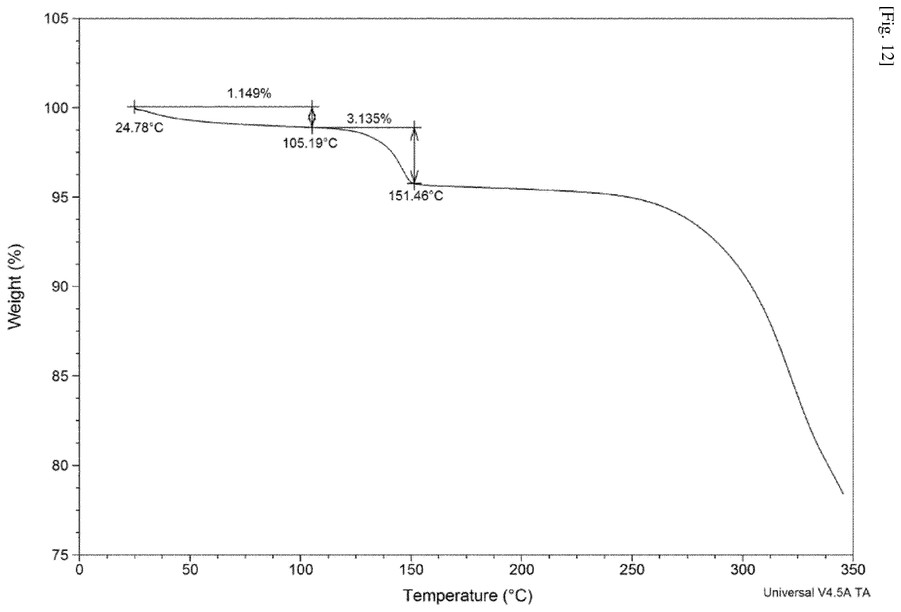

FIG. 13 is a DSC pattern analysis graph of the potassium salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid prepared in Comparative Example 4.

FIG. 14 is a graph showing results of dynamic vapor sorption (DVS) measurement of the tromethamine salt compound represented by Chemical Formula I prepared in Example 3 of the present disclosure (crystalline form 1).

FIG. 15 is a graph showing results of DVS measurement of the L-Arginine salt compound of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid prepared in Comparative Example 2.

FIG. 16 is a graph showing results of DVS measurement of the meglumine salt compound of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid prepared in Comparative Example 3.

FIG. 17 is a graph showing results of DVS measurement of the potassium salt compound of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid prepared in Comparative Example 4.

FIG. 18 is X-ray powder diffraction analysis (XRPD) graph of a tromethamine salt compound represented by Chemical Formula I prepared in Example 3 of the present disclosure (crystalline form 1), and crystalline form 1A analyzed under the conditions of 55% RH or higher at 20° C.

### BEST MODE FOR CARRYING OUT THE INVENTION

Hereinafter, Examples and the like will be described in detail to assist the under-standing of the present disclosure. However, these Examples according to the present disclosure may be modified in various other forms, and the scope of the present disclosure should not be construed as being limited to the following Examples. These Examples of the present disclosure are provided to more fully explain the present disclosure to those of ordinary skill in the art.

### <XRPD, DVS and NMR Analysis Conditions>

### 1) XRPD (X-Ray Powder Diffractometer)

The X-ray powder diffraction (XRPD) pattern was measured using DYY884-AERIS-300 manufactured by PANalytical company, and analyzed with a step size of about 0.02° over the range of about 2° to 45° in the 20 angle. The conditions of use were set as shown in Table 1 below.

TABLE 1

| Settings | Parameters |
|---|---|
| Scan Axis | Gonio |
| Scan mode | Continuous |
| Start angle (°) | 2.99 |
| End angle (°) | 45 |
| Step size (°) | 0.0217329 |
| Time per step(s) | 34.425 |
| Total time (min) | 00:04:55 |
| Generator Settings | 7.5 mA, 40 kV |
| X-ray wavelength (Å) | 1.54056 Å |
| Instrument K value | 0.5 (Kα-2/Kα-1 ratio) |

### 2) DVS (Dynamic Vapor Sorption)

Samples in amounts of about 5 to 25 mg were tested for moisture sorption/desorption profiles at 25° C. under a 0% to 90% relative humidity (RH) cycle according to the pa-rameters in Table 2 below using DVS Intrinsic 1 manufactured by Surface Measurement System Ltd.

TABLE 2

| Settings | Parameters | |
|---|---|---|
| Temperature | 25° | C. |
| Sample size | 5~25 | mg |
| Gas and flow rate | N₂, 200 | mL/minute |
| Stage time | 30 | minute |
| RH range | 0-90% | RH |
| RH step size | 10% | RH |

### 3) 1H-NMR (Nuclear Magnetic Resonance)

Approximately 3 mg of the compound was weighed into a nuclear magnetic tube and 0.5 mL deuterated dimethyl sulfoxide was added to completely dissolve the sample. The tube was put in the rotor and placed in the open position of the auto sampler and scanned with a BRUKER AVANCE III (400 MHZ).

### <Example 1> Preparation of tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid

To 500 mg of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid free base prepared according to Comparative Example 1, 5 mL of acetone was added and mixed at room temperature. To the mixture, 1.1 equivalents of tromethamine was added and reacted with reflux stirring for 1 hour. The reaction solution was cooled to room temperature and stirred for 12 hours. The obtained solid was filtered, washed with acetone, and dried to obtain 0.4 g of tromethamine salt (yield: 65.6%).

The results of NMR analysis of the tromethamine salt prepared according to Example 1 are as follows.

¹H NMR (400 MHZ, DMSO-d6): δ 8.99 (dd, Ja=2.0 Hz, Jb=0.4 Hz, 1H), 8.30 (dd, Ja=8.0 Hz, Jb=2.0 Hz, 1H), 8.19 (d, J=0.8 Hz, 1H), 7.82 (dd, Ja=8.4 Hz, Jb=1.2 Hz, 1H), 7.58-7.54 (m, 2H), 7.51 (t, Ja=16.0 Hz, Jb=8.0 Hz, 1H), 6.33 (d, J=8.0 Hz, 1H), 6.20 (d, J=7.6 Hz, 1H), 5.42 (s, 2H), 5.09-5.07 (m, 1H), 4.78-4.72 (m, 1H), 4.63-4.59 (m, 1H), 4.51-4.46 (m, 1H), 4.40-4.35 (m, 1H), 3.94 (d, J=13.2 Hz, 1H), 3.77 (d, J=13.6 Hz, 1H), 3.40 (s, 6H), 3.33-3.32 (m, 4H), 2.73-2.65 (m, 1H), 2.48-2.41 (m, 5H).

### <Example 2> Preparation of tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid

To 16 g of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid free base prepared according to Comparative Example 1, 3 mL of methanol and 15 mL of ethanol were added and mixed at room temperature. 1.05 equivalent of tromethamine was added to the mixture and reacted by stirring at 70° C. for 1 hour. The reaction solution was cooled to room temperature and stirred for 12 hours. The obtained solid was filtered, washed with ethanol, and dried to obtain 17.64 g of tromethamine salt (yield: 90.0%).

### <Example 3> Preparation of tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid

To 3.4 kg of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid free base prepared according to Comparative Example 1, 17 L of methanol and 34 L of ethyl acetate were added and mixed at room temperature. 1.05 equivalent of tromethamine was added to the mixture and reacted by stirring at 55 to 60° C. for 1 hour. The reaction solution was cooled to room temperature and stirred for 12 hours. The obtained solid was filtered, washed with ethyl acetate and dried to obtain 3.84 kg of tromethamine salt (yield: 92.31%).

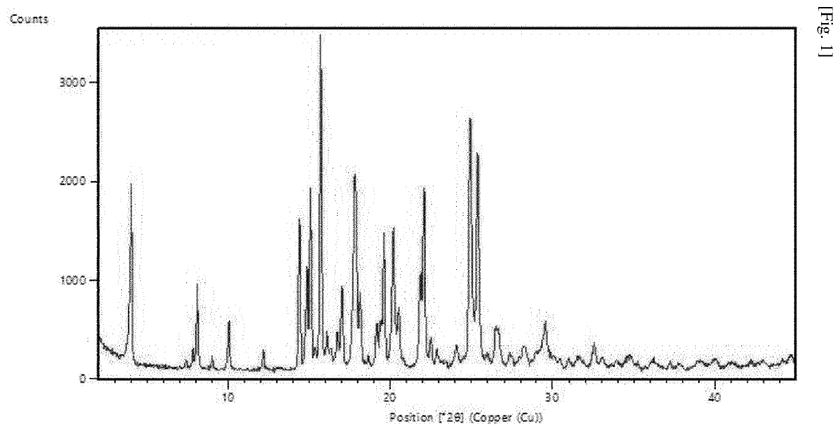

As a result of measuring XRPD for the tromethamine salt prepared in Example 3, the 2θ diffraction pattern as shown in FIG. 1 and Table 3 was confirmed, which was confirmed that the tromethamine salt prepared in Example 3 had the same crystalline form as the tromethamine salts of Examples 1 and 2. In addition, the above results showed only one crystalline form.

TABLE 3

| Peak positions | Relative |
|---|---|
| (2θ ± 0.2° 2θ) | intensity (%) |
| 4.0306 | 52.65 |
| 8.0949 | 25.24 |
| 10.0371 | 14.47 |
| 12.1710 | 6.19 |
| 14.3917 | 44.63 |
| 14.8574 | 30.86 |
| 15.0714 | 53.37 |
| 15.7076 | 100.00 |
| 17.0190 | 24.86 |
| 17.9014 | 47.73 |
| 19.5997 | 39.99 |
| 20.1847 | 41.72 |
| 21.8141 | 25.69 |
| 22.0691 | 53.87 |
| 24.9189 | 74.90 |
| 25.3857 | 63.83 |

### <Comparative Example 1> Preparation of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid free base

The(S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid free base may be prepared in the same way as disclosed in Korean Patent Laid-Open Publication No. 10-2021-0059653, and the specific method thereof is as follows.

### Step (1): Synthesis of methyl(S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylate

6-(((6-(Piperazin-1-yl)pyridin-2-yl)oxy)methyl) nicotinonitrile (233 mg), methyl(S)-2-(chloromethyl)-1-(oxetan-2-ylmethyl)-1H-benzo[d]imidazol-6-carboxylate (232 mg), and potassium carbonate (436 mg) were put in a round bottom flask, dissolved in acetonitrile (10 mL), and stirred at 60° C. for one day. After confirming the completion of the reaction by TLC, the solution was diluted with ethyl acetate, and the organic layer was washed with a saturated NaHCO₃ aqueous solution, a saturated NH₄Cl aqueous solution, and brine. Next, the organic layer was dried over anhydrous magnesium sulfate and filtered under reduced pressure to obtain a filtrate. The filtrate was concentrated under reduced pressure. The residue was purified via silica gel column chromatography (hexane/ethyl acetate) to obtain the target compound, methyl(S)-2-((4-(6-((5-cyanopyridin-2-yl)methoxy)pyridin-2-yl)piperazin-1-yl)methyl)-1-(oxe tan-2-ylmethyl)-1H-benzo[d]imidazol-6-carboxylate (390 mg, yield 89%) as a clear syrup.

### Step (2): Synthesis of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid

The compound (387 mg) obtained in step (1) was put into a round bottom flask, dissolved in acetonitrile (10 mL), and stirred. A 1.0 M TBD aqueous solution (1.4 mL) was added dropwise while stirring. Purified water (0.6 mL) was added to the mixture and stirred at 60° C. for one day. After confirming the completion of the reaction by TLC, the mixture was neutralized to pH 7 with 1N HCl aqueous solution, extracted with a 10% DCM/MeOH solution, dried over anhydrous magnesium sulfate, and filtered under reduced pressure to obtain a filtrate. The filtrate was concentrated under reduced pressure. The residue was purified by silica gel column chromatography (DCM/MeOH) to obtain the final compound as a pale green solid (225 mg, yield 60%).

### <Comparative Example 2> Preparation of L-arginine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid

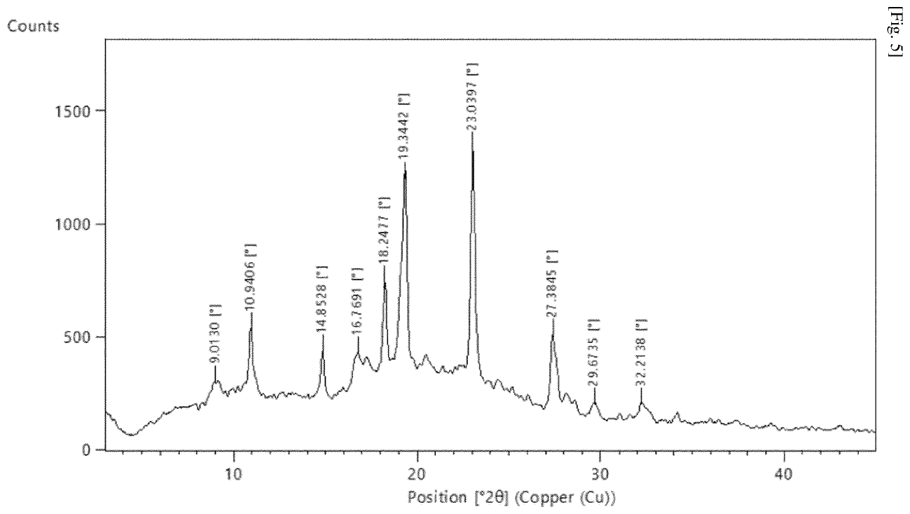

To 2 mL vial containing 20 mg of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid free base, 1 mL of acetonitrile was added, and the resulting mixture was mixed at room temperature for 5 seconds. To the mixture, 1.1 equivalents of L-Arginine was added, and a ThermoMixer C (Eppendorf) was employed for a heating and cooling cycle. After temperature cycling, the suspension was centrifuged to obtain a precipitate, and the precipitate obtained was dried under vacuum conditions at room temperature to obtain an L-arginine salt. XRPD analysis was performed on the L-arginine salt, and results thereof are shown in FIG. 5.

### <Comparative Example 3> Preparation of meglumine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid

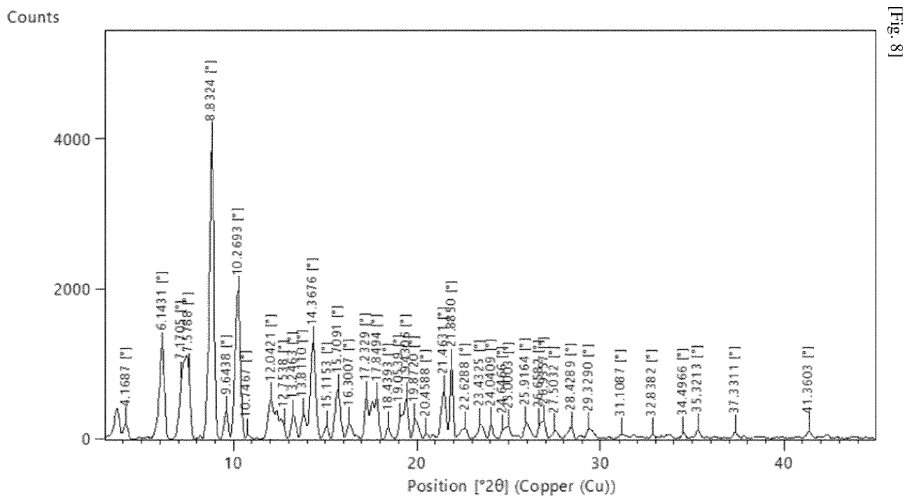

To 2 mL vial containing 20 mg of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid free base, 1 mL of acetonitrile was added, and the resulting mixture was mixed at room temperature for 5 seconds. To the mixture, 1.1 equivalents of meglumine was added, and the ThermoMixer C (Eppendorf) was employed for the heating and cooling cycle. After temperature cycling, the suspension was centrifuged to obtain a precipitate, and the precipitate obtained was dried under vacuum conditions at room temperature to obtain a meglumine salt. XRPD analysis was performed on the meglumine salt, and results thereof are shown in FIG. 8.

### <Comparative Example 4> Preparation of potassium salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid

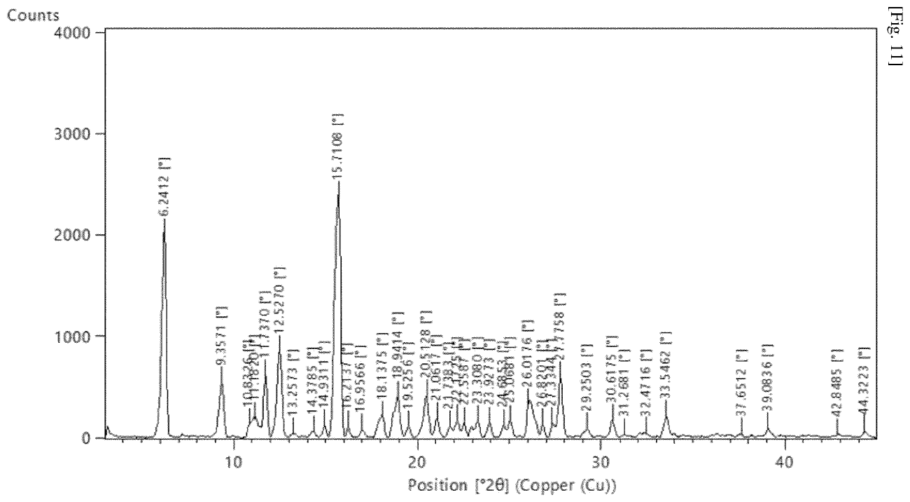

To 2 mL vial containing 20 mg of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid free base, 1 mL of acetonitrile was added, and the resulting mixture was mixed at room temperature for 5 seconds. To the mixture, 1.1 equivalents of potassium hydroxide (KOH) were added, and the ThermoMixer C (Eppendorf) was employed for the heating and cooling cycle. After temperature cycling, the suspension was centrifuged to obtain a precipitate, and the precipitate obtained was dried under vacuum conditions at room temperature to obtain a potassium salt. XRPD analysis was performed on the potassium salt, and results thereof are shown in FIG. 11.

### <Experimental Example 1> Thermogravimetric (TGA) Analysis

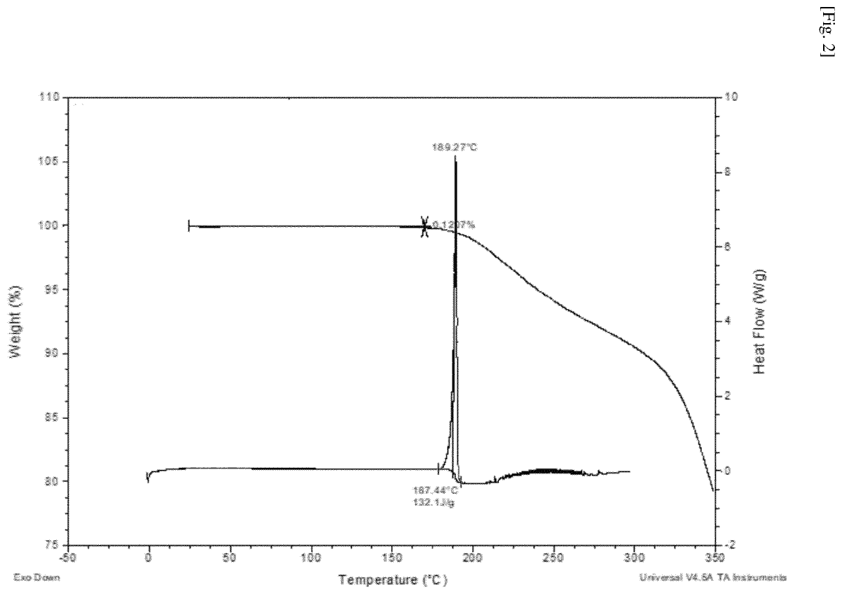

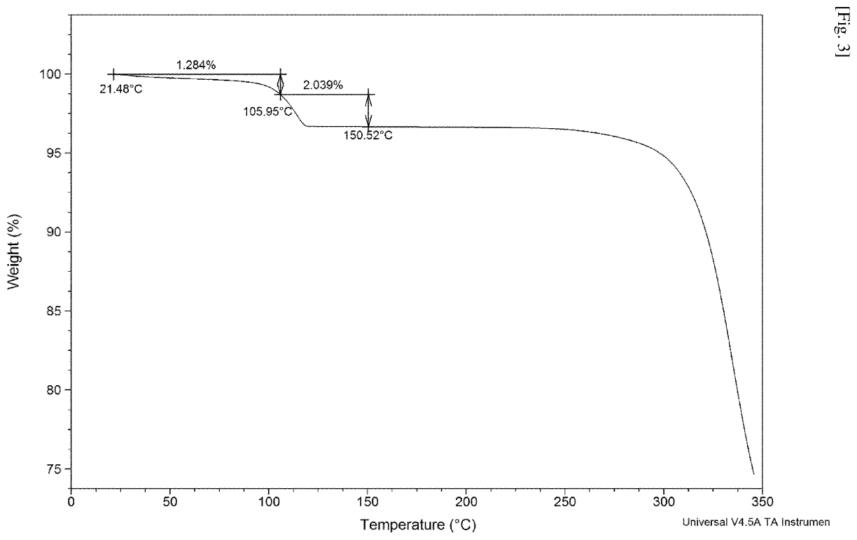

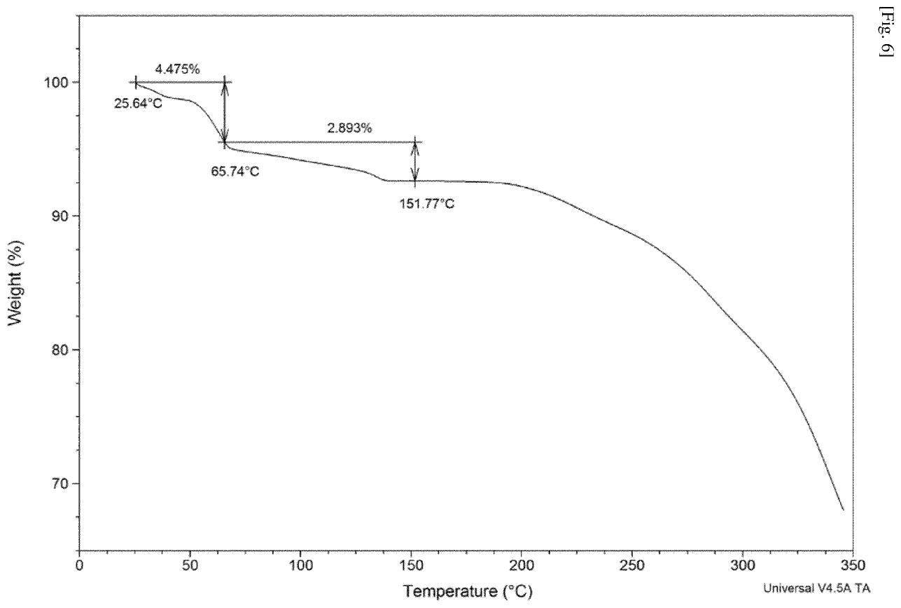

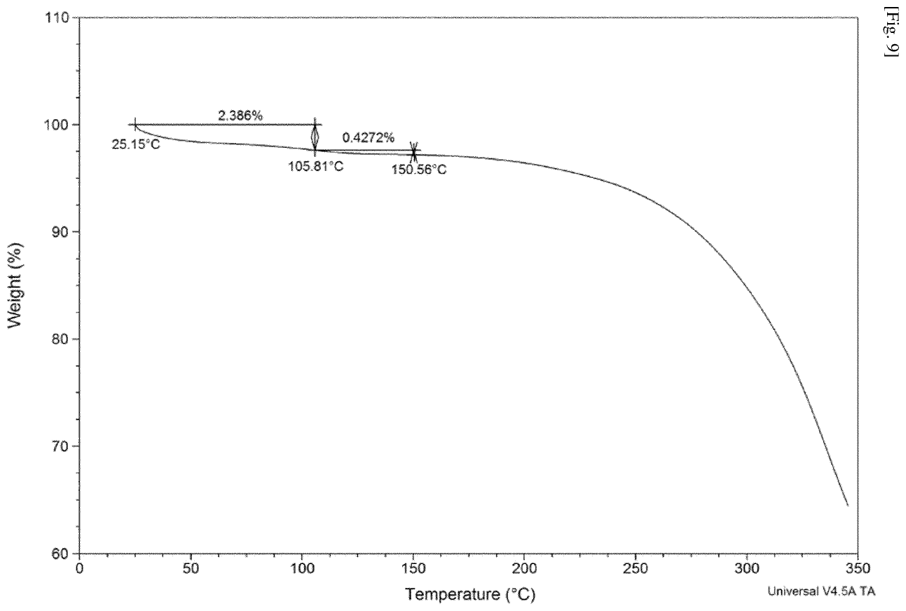

Thermogravimetric (TGA) analysis was performed on the tromethamine salt prepared in Example 3, the free base prepared in Comparative Example 1, the L-arginine salt prepared in Comparative Example 2, the meglumine salt prepared in Comparative Example 3, and the potassium salt prepared in Comparative Example 4. Thermogravimetric analysis was performed from 20° C. to 350° C. at a heating rate of 10° C./min under nitrogen conditions using TGA Q50 manufactured by TA Instruments, Inc. The weight loss at about 170° C. or less for each salt was measured, and results thereof are shown in FIG. 2 (tromethamine salt), FIG. 3 (free base), FIG. 6 (L-arginine salt), FIG. 9 (meglumine salt), and 12 (potassium salt), and summarized in Table 4 below.

TABLE 4

| | | Weight loss (%) at |
|---|---|---|
| Test Compound | | about 170° C. or less |
| Example 3 | Tromethamine salt | 0.1207 |
| Comparative | Free base | 3.323 |
| Example 1 | | |
| Comparative | L-arginine salt | 7.368 |
| Example 2 | | |
| Comparative | Meglumine salt | 2.8132 |
| Example 3 | | |
| Comparative | Potassium salt | 4.284 |
| Example 4 | | |

As could be seen from the above results, the free base of Comparative Example 1 and other salts of Comparative Examples 2 to 4 showed a weight loss caused by containing water or organic solvent, but the tromethamine salt according to the present disclosure showed little weight loss. Therefore, it could be confirmed that the tromethamine salt according to the present disclosure had excellent physicochemical properties that did not contain water or organic solvents.

### <Experimental Example 2> Differential Scanning Calorimetry (DSC) Analysis

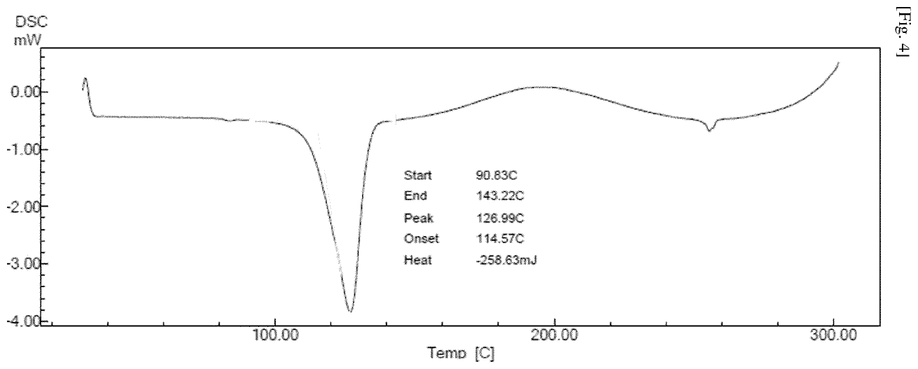

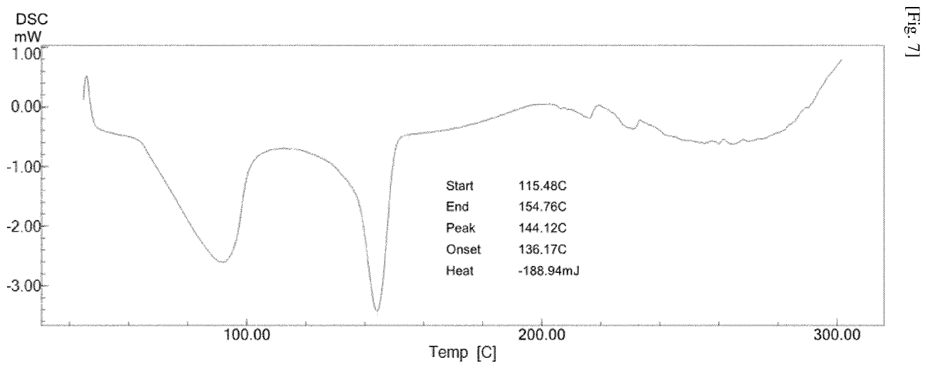

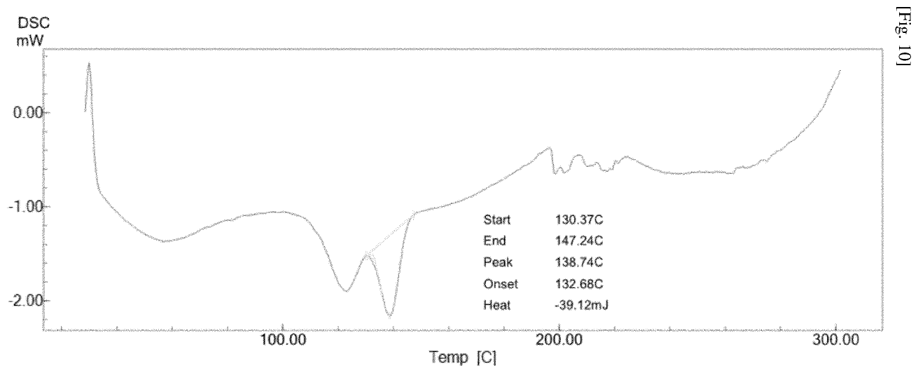

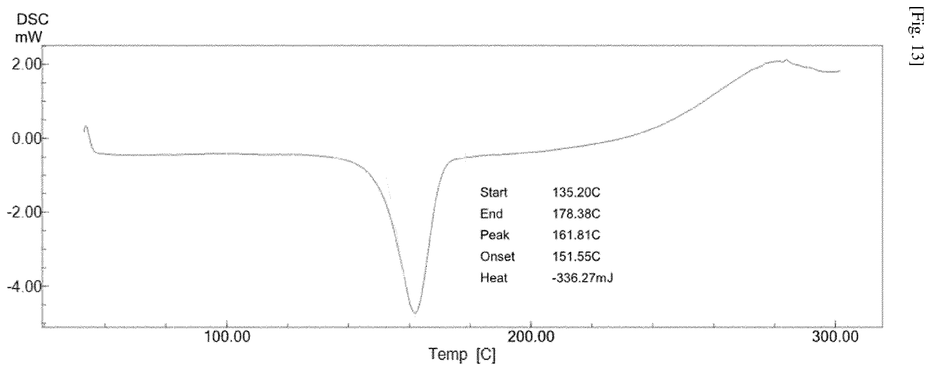

Differential scanning calorimetry (DSC) analysis was performed on the tromethamine salt prepared in Example 3, the free base prepared in Comparative Example 1, and other salts prepared in Comparative Examples 2 to 4. Specifically, using a DSC analyzer, DSC was measured in an airtight pan at a scan rate of 10° C./min over the range of 30° C. to 300° C. under a nitrogen purge. Accordingly, the DSC results of the tromethamine salt prepared in Example 3 are shown in FIG. 2, the DSC results of the free base of Comparative Example 1 are shown in FIG. 4, and the DSC results of other salts obtained in Comparative Examples 2 to 4 are shown in FIGS. 7, 10 and 13, respectively, and summarized in Table 5 below.

TABLE 5

| Test Compound | | Onset(° C.) | Peak(° C.) |
|---|---|---|---|
| Example 3 | Tromethamine salt | 187.44 | 189.27 |
| Comparative | Free base | 114.57 | 126.99 |
| Example 1 | | | |
| Comparative | L-arginine salt | 136.17 | 144.12 |
| Example 2 | | | |
| Comparative | Meglumine salt | 132.68 | 138.74 |
| Example 3 | | | |
| Comparative | Potassium salt | 151.55 | 161.81 |
| Example 4 | | | |

As could be confirmed in Table 5, the(S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid free base had the disadvantage of poor thermodynamic stability due to a low melting point. On the other hand, it could be confirmed that the tromethamine salt of the present disclosure had a significantly higher melting point than other salts and the free base obtained in Comparative Examples 2 to 4, thereby exhibiting excellent thermodynamic stability.

### <Experimental Example 3> Hygroscopicity Test

### (1) 25° C. and 90% RH Condition

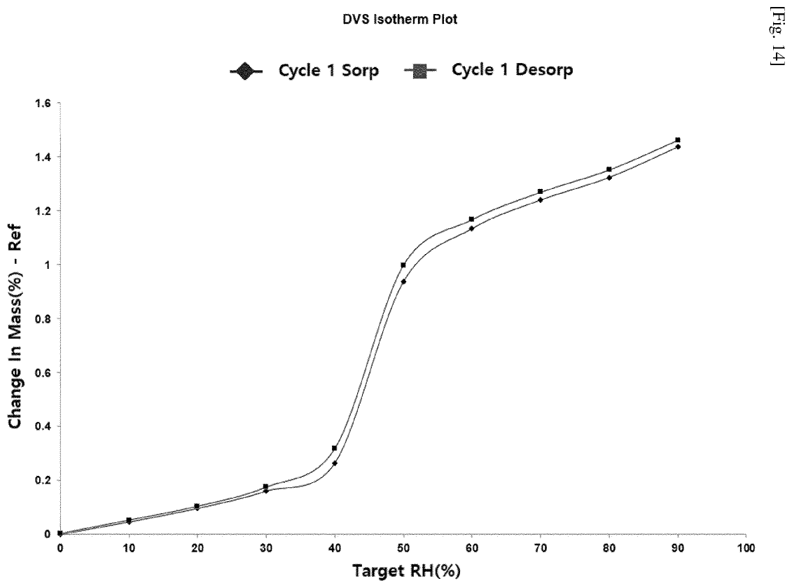

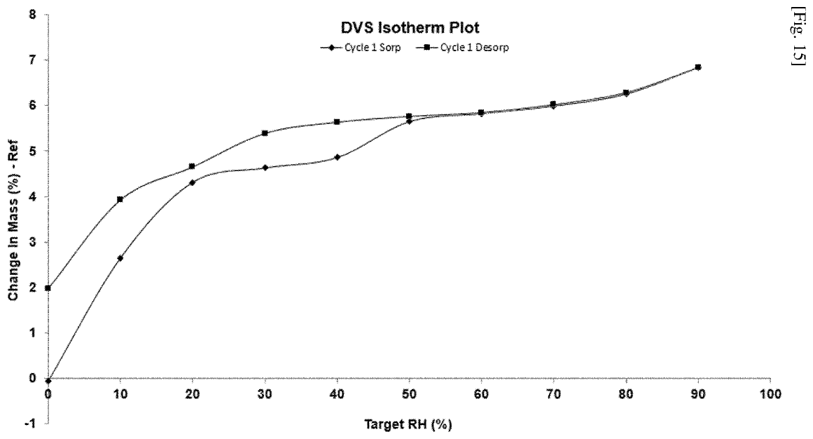

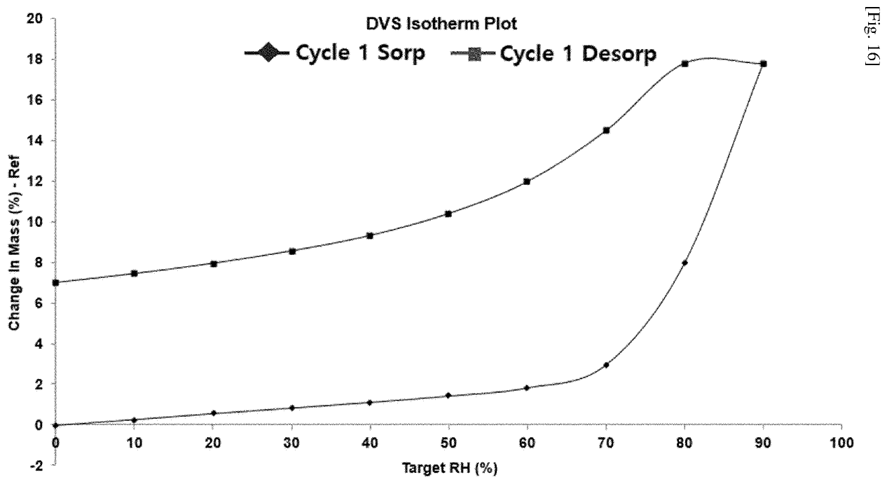

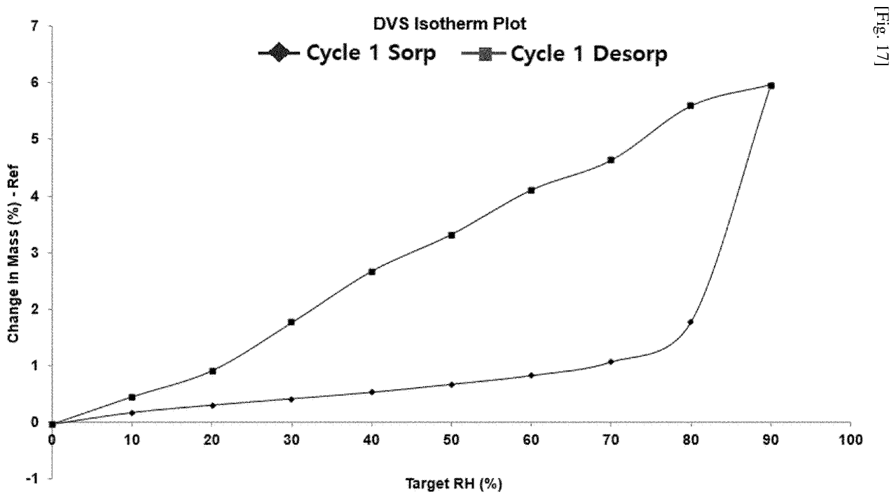

Hygroscopicity was measured for the tromethamine salt prepared in Example 3, the L-arginine salt prepared in Comparative Example 2, the meglumine salt prepared in Comparative Example 3, and the potassium salt prepared in Comparative Example 4. Results thereof are shown in FIGS. 14 to 17, respectively, and the results of the weight percent gain according to moisture sorption are shown in Table 6 below.

TABLE 6

| | | Weight percent |
|---|---|---|
| Test Compound | | gain (%) |
| Example 3 | Tromethamine salt | 1.44 |
| Comparative | L-arginine salt | 6.84 |
| Example 2 | | |
| Comparative | Meglumine salt | 17.77 |
| Example 3 | | |
| Comparative | Potassium salt | 5.97 |
| Example 4 | | |

As could be confirmed in FIGS. 14 to 17 and Table 6, the tromethamine salt showed a weight change of about 1.44% when measuring the hygroscopicity through DVS at 25° C. and 90% humidity, and exhibited significantly lower hygroscopicity compared to L-arginine salt, meglumine salt and potassium salt. Therefore, it was confirmed that the novel tromethamine salt of the present disclosure had excellent storage stability.

### (2) 25° C. and 80% RH Condition

Under the hygroscopicity test conditions of the European Pharmacopoeia (EP), the tromethamine salt prepared in Example 3 and the free base prepared in Comparative Example 1 were stored for 24 hours at a temperature of 25° C. and a relative humidity of 80%, and then the mass change of the sample due to moisture sorption was measured.

Results thereof are shown in FIG. 7.

TABLE 7

| | | Weight percent |
|---|---|---|
| Test Compound | | gain (%) |
| Example 3 | Tromethamine salt | 0.22 |
| Comparative | Free base | 2.7 |
| Example 1 | | |

As could be confirmed in Table 7, the tromethamine salt of the present disclosure had a coefficient of moisture absorption of 0.22%, and exhibited remarkably excellent non-hygroscopicity than the(S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid free base.

Therefore, it was confirmed that the tromethamine salt of the present disclosure had remarkably excellent storage stability compared to the free base and was suitable for formulation.

### <Experimental Example 4> Evaluation of Solubility in Bio-Relevant Media

Solubility in water and bio-relevant media, i.e., stimulated gastric fluid (SGF), fasted state simulated intestinal fluid (FaSSIF), and fed state simulated intestinal fluid (FeSSIF) of the free base and the tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid was confirmed.

Buffers for the bio-relevant media were prepared as follows.

Water: Laboratory Milli-Q purified water.

SGF (Stimulated Gastric Fluid): 2.0 g of sodium chloride and 7 mL of hydrochloric acid dissolved in 1000 mL of water was used.

FaSSIF (Fasted State Simulated Intestinal Fluid): A commercial product purchased from Bio-Relevant Company and manufactured according to instructions was used.

FeSSIF (Fed State Simulated Intestinal Fluid): A commercial product purchased from Bio-Relevant Company and prepared according to instructions was used.

For the experiment, about 2 mg of compound (2 mg as free base form) was weighed into each glass vial and then 1 mL of medium was added (final concentration of 2 mg/mL). Then, the sample was continuously stirred for 24 hours in a magnetic stirrer at a speed of 5000 rpm at room temperature. Then, the pH was measured and the mixture was filtered using MultiScreen®_{HTS}-HV 96-well plate (Merck; 0.45 μm hydrophilic low protein binding Durapore® membrane). Solubility was analyzed using HPLC. Experimental results thereof are shown in Table 8.

TABLE 8

| | Example 3 | | Comparative Example 1 | |
|---|---|---|---|---|
| | Tromethamine salt | | Free base | |
| | Solubility | Final | Solubility | Final |
| Medium | (mg/mL) | pH | (mg/mL) | pH |
| SGF (pH 2.0) | 0.710 | 1.90 | 0.850 | 1.74 |
| FaSSIF (pH 6.5) | >2.00 | 6.50 | 0.160 | 6.49 |
| FeSSIF (pH 5.8) | 0.336 | 5.85 | 0.063 | 5.70 |
| Water | >2.00 | 7.21 | 0.106 | 6.64 |

As could be confirmed in Table 8 above, the tromethamine salt showed excellent solubility in all of SGF, FaSSIF, and FeSSIF, which regulate the pH similar to the in-vivo stomach, small intestine before meals, and small intestine after meals, but in particular exhibited very good solubility under fasted state simulated intestinal fluid (FaSSIF) conditions that mimic the small intestine where most drug absorption occurs. This action shows that the tromethamine salt is able to show high solubility regardless of meal, and to have high absorption compared to free base, thereby having excellent bioavailability, and the like.

### <Experimental Example 5> Drug Stability Evaluation

The free base and the tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid were weighed into each vial and stored under stress conditions to investigate stability.

The stress conditions are as follows:
high temperature open room condition: 80° C.;

Samples were analyzed by XRPD and HPLC at the onset (Day 0) and week 1 of storage. HPLC results thereof are shown in Table 9.

TABLE 9

| | | Chemical stability | |
|---|---|---|---|
| | | by HPLC purity % | |
| Test Compound | | Onset (Day 0) | 80° C. (Week 1) |
| Comparative | Free base | 97.84 | 96.70 |
| Example 1 | | | |
| Example 3 | Tromethamine salt | 97.99 | 97.32 |

As could be seen in Table 9, the tromethamine salt according to the present disclosure had a purity change rate of less than 1% under harsh conditions, showing remarkably excellent stability compared to that of the free base. In addition, it was confirmed that the tromethamine salt according to the present disclosure was a very stable material under harsh conditions that did not show any change in the XRPD pattern and had no change in appearance.

### <Experimental Example 6> Analysis of Crystal Form Under Various Relative Humidity Conditions (Reversible Adsorption/Desorption)

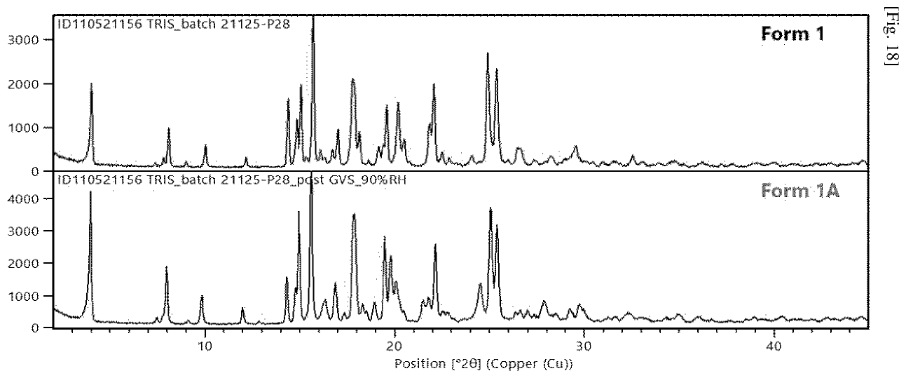

The XRPD analysis of the crystalline form 1 of the tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid was performed under the conditions of varying relative humidity from 15% RH to 90% RH at 20° C., and the changes in the crystalline form according to the relative humidity were confirmed. The results are shown in FIG. 18, Table 10 and 11. Table 10 shows the crystalline form while changing the relative humidity from 15% RH to 60% RH, and Table 11 shows the crystalline form during desorption of from 50% RH to 15% RH.

TABLE 10

| | Relative humidity (% RH, 20° C.) | | | | | | | | | |
|---|---|---|---|---|---|---|---|---|---|---|
| | 15 | 20 | 25 | 30 | 35 | 40 | 45 | 50 | 55 | 60 |
| Crystalline | Form | Form | Form | Form | Form | Form | Form | Form | Form | Form |
| Form | 1 | 1 | 1 | 1 | 1 | 1 | 1 | 1 | 1A | 1A |

TABLE 11

| Relative | | | | |
|---|---|---|---|---|
| humidity | | | | |
| (% RH, 20° C.) | 50 | 40 | 30 | 15 |
| Crystalline | intermediate | Form 1 | Form 1 | Form 1 |
| Form | reflections between | | | |
| | Form 1 and Form 1A | | | |

As could be confirmed in FIG. 18, the crystalline form 1A has the 2θ diffraction pattern (2θ±0.2°) shown in Table 3 above, and further comprises diffraction peaks at 2-theta (2θ) angles of at 19.8019±0.2 and 24.5314±0.2.

As could be confirmed in Table 10 and 11, the tromethamine salt of the present disclosure maintained the form of crystalline Form 1 under conditions 15% RH or more and less than 55% RH at 20° C.

However, a change in the XRPD peak exhibited under the conditions of 55% RH or more at 20° C. (formation of form 1A), but when desorption was performed under the condition of 15% RH or higher and less than 55% RH at 20° C., form 1A was reverted to the form of crystalline form 1.

The above result shows that the crystalline Form of tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid is a non-stoichiometric hydrate, only expansion and contraction of the lattice during adsorption and desorption of water, and there is no collapse of the lattice structure.

1-21. (canceled)

22. A tromethamine salt of (S)-2-((4-(6-((5-cyanopyridin-2-yl)methoxy)pyridin-2-yl)piperazin-1-yl)methyl)-1-(oxetan-2-ylmethyl)-1H-benzo[d]imidazol-6-carboxylic acid represented by the following Chemical Formula I:

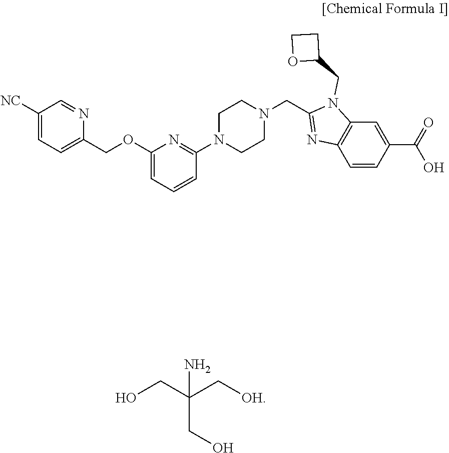

23. The tromethamine salt of claim 22, wherein the tromethamine salt has a thermogravimetric analysis (TGA) pattern showing a weight loss of less than 0.2 wt % at 170° C. or less.

24. The tromethamine salt of claim 22, wherein the tromethamine salt has an endothermic transition peak value at 174 to 204° C. in a differential scanning calorimetry (DSC) graph.

25. The tromethamine salt of claim 22, wherein the tromethamine salt has an endothermic transition peak value at 189±2° C. in a differential scanning calorimetry (DSC) graph.

26. The tromethamine salt of claim 22, wherein the tromethamine salt is a crystalline form.

27. The tromethamine salt of claim 26, wherein the crystalline form comprises, in an X-ray powder diffraction (XRPD) graph, at least three diffraction peaks at 2-theta (2θ) angle values selected from 4.03±0.2, 8.09±0.2, 10.04±0.2, 15.07±0.2, 15.71±0.2, 17.90±0.2, 19.60±0.2, 22.07±0.2, 24.92±0.2, and 25.39±0.2.

28. The tromethamine salt of claim 27, wherein the crystalline form comprises, in the XRPD graph, diffraction peaks at 2-theta (2θ) angle values of 4.03=0.2, 8.09±0.2, 10.04±0.2, 15.07±0.2, 15.71±0.2, 17.90±0.2, 19.60±0.2, 22.07±0.2, 24.92±0.2, and 25.39±0.2.

29. The tromethamine salt of claim 27, wherein the crystalline form further comprises, in the XRPD graph, one or more diffraction peaks at 2-theta (2θ) angle values selected from 12.17±0.2, 14.39±0.2, 17.02±0.2, 20.18±0.2, and 21.81=0.2.

30. The tromethamine salt of claim 27, wherein the crystalline form further comprises, in the XRPD graph, one or more diffraction peaks at 2-theta (2θ) angle values selected from 19.80=0.2 and 24.53=0.2.

31. The tromethamine salt of claim 22, wherein the tromethamine salt is a crystalline form 1,
wherein the crystalline form 1 comprises, in the XRPD graph, one or more diffraction peaks at 2-theta (2θ) angle values selected from 4.03±0.2, 8.09±0.2, 10.04=0.2, 15.07=0.2, 15.71±0.2, 17.90±0.2, 19.60=0.2, 22.07=0.2, 24.92=0.2, and 25.39=0.2.

32. The tromethamine salt of claim 31, wherein the crystalline form 1A reverts to the crystalline form 1 upon desorption of water by changing the relative humidity to less than 55% RH at 20° C.

33. A pharmaceutical composition comprising the tromethamine salt according to claim 22 and a pharmaceutically acceptable carrier.

34. A method of treating a metabolic disease in a subject in need thereof, comprising administering to the subject a therapeutically effective amount of the tromethamine salt according to claim 22.

35. The method of claim 34, wherein the metabolic disease is selected from diabetes mellitus, idiopathic type 1 diabetes, latent autoimmune diabetes in adults (LADA), early onset type 2 diabetes (EOD), youth-onset atypical diabetes (YOAD), maturity-onset diabetes of the young (MODY), malnutrition-related diabetes, gestational diabetes, hyperglycemia, insulin resistance, liver insulin resistance, impaired glucose tolerance, diabetic neuropathy, diabetic nephropathy, kidney disease, diabetic retinopathy, visceral fat accumulation, sleep apnea, obesity, eating disorders, dyslipidemia, hyperinsulinemia, non-alcoholic fatty liver disease (NAFLD), cardiovascular disease, atherosclerosis, peripheral vascular disease, hypertension, congestive heart failure, myocardial infarction, stroke, hemorrhagic stroke, ischemic stroke, traumatic brain injury, pulmonary hypertension, restenosis after angioplasty, intermittent claudication, postprandial lipidemia, metabolic acidosis, ketosis, arthritis, osteoporosis, Parkinson's disease, left ventricular hypertrophy, peripheral arterial disease, loss of vision, cataracts, glomerulosclerosis, chronic renal failure, metabolic syndrome, Syndrome X, premenstrual syndrome, angina, thrombosis, transient ischemic attack, vascular restenosis, impaired glucose metabolism, symptoms of impaired fasting blood sugar, hyperuricemia, gout, erectile dysfunction, psoriasis, foot ulcers, ulcerative colitis, hyper-apo B lipoproteinemia, Alzheimer's disease, schizophrenia, cognitive impairment, inflammatory bowel disease, short bowel syndrome, Crohn's disease, colitis, irritable bowel syndrome, and polycystic ovary syndrome.

36. The method of claim 35, wherein the non-alcoholic fatty liver disease (NAFLD) is selected from steatosis, non-alcoholic steatohepatitis (NASH), fibrosis, cirrhosis, and hepatocellular carcinoma.

37. The tromethamine salt of claim 31, wherein the tromethamine salt is a crystalline form 1A, wherein the crystalline form 1A comprises, in the XRPD graph, one or more diffraction peaks at 2-theta (2θ) angle values selected from 19.80±0.2 and 24.53±0.2, in addition to the diffraction peaks at 2-theta (2θ) angle values of the crystal form 1.

38. The tromethamine salt of claim 22, wherein the tromethamine salt is a crystalline form 1, which changes to a crystalline form 1A upon exposure to a relative humidity of 55% RH or higher at 20° C.
